(19) United States
(12) Patent Application Publication
(10) Pub. No.: US 2006/0279699 A1
(43) Pub. Date: Dec. 14, 2006

(54) Wavefront fusion algorithms for refractive vision correction and vision diagnosis

(75) Inventor: Junzhong Liang, Fremont, CA (US)

Correspondence Address: JUNZHONG LIANG, 45 KOOTENAI DRIVE, FREMONT, CA, 94539, US

(73) Assignee: Advanced Vision Engineering, inc, Fremont, CA (US)

(21) Appl. No.: 11/432,273

(22) Filed: May 10, 2006

Related U.S. Application Data
(60) Provisional application No. 60/690,601, filed on Jun. 14, 2005.

(51) Int. Cl. A61B 3/00

(52) U.S. Cl. 351/246

(57) ABSTRACT

Accommodation-Free Wavefront, wave aberration of an eye at the far accommodation points, is determined using a wavefront fusion algorithm by obtaining a wave aberration of an eye from a wavefront measurement, obtaining a manifest refraction of an eye at a far accommodation point according to a conventional subjective refraction, and determining a wave aberration of the eye at its far accommodation point based on a combination of the manifest refraction and the measured wave aberration of the same eye. Wave aberration of an eye at the far accommodation points enable accommodation-free wavefront-guided vision corrections as well as comprehensive vision diagnosis of human vision based on a true-vision wavefront. True-Vision wavefront is determined from the accommodation-free wavefront with removal of a refractive prescription of a correction lens if the lens is used for a sphero-cylindrical correction.

### CROSS-REFERENCES TO RELATED INVENTIONS

The present invention claims priority to the provisional U.S. patent application 60/690,601, titled “Methods and apparatus for improving and evaluating wavefront-guide vision corrections,” filed on Jun. 14, 2005 by Liang. The disclosures of these related applications are incorporated herein by reference.

### TECHNICAL FIELD

This application relates to systems and methods for refractive vision corrections, in particular, for determining an accommodation-free wavefront of an eye for wavefront-guide vision corrections and a true-vision wavefront of an eye for comprehensive vision diagnosis.

### BACKGROUND

Wavefront-guide technology, or customized vision correction, is becoming a new frontier for vision and ophthalmology because it offers the capability to manipulate high-order aberrations in the eye. Wavefront technology will reshape the eye care industry by enabling customized design of laser vision corrections, contact lenses, intro-ocular lenses, and even spectacles.

Wavefront technology is based primarily on the measurement of eye's wave aberration using a wavefront sensing device. One popular technique of wavefront sensing is to use a Hartmann-Shank wavefront sensor as disclosed in “Objective measurement of wave aberrations of the human eye with the use of a Hartmann-Shack wave-front sensor,” J. Opt. Soc. Am. A, vol. 11, no. 7, p. 1949 (July 1994) by Liang et al. Wave aberration in the eye can also be measured with other devices such as ray tracing aberrometers, Talbot Interferometry based aberrometers, and phase retrieval methods.

Wavefront sensors measure all aberrations in the eye including focus error, cylindrical error (astigmatism), spherical aberration, coma and a host of other high order aberrations. Focus error and cylindrical error form a sphero-cylindrical error that can be corrected by convention lenses. Because sphero-cylindrical errors are also measured in manifest refraction in optometric practice, wavefront refractions of sphero-cylindrical errors in wavefront measurements are often validated with the manifest refractions.

FIG. 1 shows a comparison between the wavefront refraction determined from wavefront measurements and the conventional manifest refraction determined with a phoroptor. Spherical equivalents (SE), defined as the sum of the spherical power and one half of the cylindrical power, from the manifest refraction (horizontal axis) and from the wavefront refraction (vertical axis) are plotted against each other for more than 100 normal eyes. Generally speaking, the sphero-cylindrical correction determined from the wavefront sensor agrees well with the manifest refraction determined with phoroptors. However, it is also evident that about 15% eyes have a difference greater than 0.5 Dioptors, indicated as those points above or below the dotted lines. An error of one-half Dioptors is significant for vision corrections.

Mismatches between the manifest refraction and the wavefront refraction are due to a number of factors, including the differenced in controlling accommodation of the tested eye in the manifest refraction and in the wavefront sensing, dependence of the conventional sphero-cylindrical correction on the high-order aberrations in the eye, and perceptional preferences of an eye dictated by retinal image processing.

Manifest correction has been proven effective for refractive corrections of focus error and astigmatism for over a century. The discrepancy between the manifest refraction and the wavefront refraction causes problems in using the wavefront data for wavefront-guided vision corrections. Questions were often raised on the accuracy and reliability of wave aberration of eye measured by wavefront sensors.

Attempts were made to find improved algorithms for the calculation of wavefront refractions as disclosed in U.S. Pat. No. 6/511,180, issued Jan. 28, 2003, for “Determination of ocular refraction from wave aberration data and design of optimum customized correction”; U.S. Pat. No. 6/808,266, issued Oct. 26, 2004, for “Objective manifest refraction”; and U.S. patent application Ser. No. 20040145702, filed Jul. 29, 2004, for “Method for determining refractive corrections from wavefront measurements.” These methods are designed to address the issue of dependence of the sphero-cylindrical correction on the high-order aberrations in the eye, but cannot solve the mismatch problem because they did not address the issues of accommodation control and the perceptional preference.

Subject refinement of wavefront measurement was disclosed in U.S. Pat. No. 6/688,745, issued Feb. 10, 2004 by Ross et al. The method of subjective refinement utilizes a closed-loop control of refractive corrections, and uses patient's response as the feedback for determining the end-point for the wavefront correction. The subjective refinement can be effective for an adaptive optics system to create a sharp retinal image for which the exact accommodation state of the tested eye is not important. It however has at least three disadvantages when used for practical refractive corrections. First, Ross's method involves in an expensive adaptive optics system to address the dependence of the conventional sphero-cylindrical error on the high-order aberrations in the eye. Second, it involves in a complicated validation process to address the issue of perceptional preference. Third, Ross's method did not address the issue of accommodation control to obtain a wavefront of an eye at the far accommodation point. Obtaining a wavefront of eye at the far accommodation point is critically important for practical vision correction because refractive vision corrections are usually designed to achieve a best corrected image quality for an eye at the far accommodation point for the largest effective focus range possible.

Without having an effective method to deal with mismatches between the subjective manifest refraction and the objective wavefront refraction, a common approach to mitigate the discrepancy between the manifest refraction and the wavefront refraction is shown in FIG. 2. The wavefront refraction 202 and the manifest refraction 203 of an eye are compared in terms of spherical equivalent. If the difference between the manifest refraction and the wavefront refraction 204 is less than a threshold value, say about 0.5D, wave aberration measured by a wavefront sensor is considered accurate and a wavefront-guided treatment will be performed based on the wavefront measured by wavefront sensor alone. If the difference is greater than the threshold value, the wavefront measurement is considered not accurate enough for a wavefront-guided treatment.

Although the approach in FIG. 2 provides reasonable mitigation to the mismatch problem, there are, however, at least three fundamental issues. First, it is likely that one out of eight eyes may not qualify for wavefront-guided vision correction because of a large discrepancy between the manifest and the wavefront refractions. Second, the discrepancy can cause over-corrections or under-corrections and leads to a high re-treatment rate when the eye is not accommodate at its far point during a wavefront measurement. The discrepancy between the manifest and the wavefront refraction will be transferred to a wrong correction. Third, the discrepancy could cause reduction in productivity because more wavefront measurements will be performed in order to make wavefront refraction to match to the manifest refraction within the specified threshold.

In addition to causing problem for wavefront-guide vision corrections, the discrepancy between the manifest refraction and the wavefront refraction also causes problems for vision evaluation. With an uncertainty in the sphero-cylindrical correction, vision evaluation using the wavefront data will be problematic because residual focus errors and cylindrical errors can be more important than the high-order aberrations in degrading vision performance. Vision evaluation of an eye can be reliable only if all aberrations in the eye are true and are reliably measured, including not only the high-order aberrations but also the low-order aberrations such as the focus error and the cylindrical error.

In light of the forgoing, it is readily apparent that a need exists in the art to provide a wavefront technology that can solve the mismatch problem between the manifest refraction and the wavefront refraction from wavefront sensing. More particularly, a need exists in the art to provide an effective method for determining all aberration of the eye at its far accommodation point, or an Accommodation-Free Wavefront, because refractive corrections as well as refractive vision evaluation of the eye are based on performance of human vision at eye's far accommodation point.

### SUMMARY

Implementations of the method may include one or more of the following. In one aspect, the present invention relates to a method of wavefront fusion for determining a wave aberration of an eye at its far accommodation point, or an Accommodation-Free Wavefront, the method comprising:

obtaining a wave aberration of an eye from a wavefront measurement;

obtaining a manifest refraction of the eye at the far accommodation point;

determining a wave aberration of the eye at its far accommodation point based on a combination of the manifest refraction and the measured wave aberration of the same eye.

In another aspect, a method of accommodation-free wavefront-guided vision correction comprises:

obtaining a wave aberration of an eye from a wavefront measurement;

obtaining a manifest refraction of the eye at the far accommodation point;

determining an accommodation-free wavefront of the eye based on a combination of the manifest refraction and the wave aberration of the eye;

correcting an optical error of an eye based on the accommodation-free wavefront.

In yet another aspect, a method of comprehensive vision diagnosis based on a true-vision wavefront comprises:

obtaining a wave aberration of an eye from a wavefront measurement;

obtaining a manifest refraction of the eye at the far accommodation point if the eye is myopic or hyperopic;

obtaining a refractive prescription of a true correction lens if a conventional sphero-cylindrical correction is involved for a refractive correction;

calculating an true-vision wavefront of the eye based on the measured wave aberration, the manifest refraction, and the refractive prescription if a correction lens is involved;

calculating at least one image quality parameter based on the true-vision wavefront of the eye for refractive vision diagnosis.

The algorithm of wavefront fusion provides an intelligent way for determine an wave aberration of an eye at the far accommdation point by combining the advantages of the wavefront technology that offers all aberrations in the eye and the manifest refraction at the far accommdation point that has been clinically effective for over a century but is limited to the spherical and cylindrical errors.

Embodiments may include one or more of the following advantages. First, the invention method provides Aberration-Free Wavefront for wavefront guided vision corrections with refractive lasers, contact lenses, intro-ocular lenses and spectacles. Vision corrections with an Aberration-Free Wavefront will allows wavefront treatments of eyes that may have significant discrepancy between a manifest refraction and a wavefront refraction, will eliminate re-treatments of eye that does not accommodates at the far point during a wavefront measurement, and make a wavefront treatment physician-independent. No individual physician adjustment is needed if the manifest refraction and the wavefront refraction of an eye are different. Second, the present invention enables reliable vision evaluation based on a TrueVision Wavefront. The True-Vision wavefront of eye is a combination of an accommodation-free wavefront plus a true refractive prescription if a sphero-cylindrical lens is used for vision correction. It provides an accurate representation of high-order aberrations as welll as the low-order sphero-cylindrical correction.

The details of one or more embodiments are set forth in the accompanying drawings and in the description below. Other features, objects, and advantages of the invention will become apparent from the description and drawings, and from the claims.

### DRAWING DESCRIPTIONS

FIG. 1 shows a comparison between the manifest refraction and the wavefront refractions of sphero-cylindrical errors for more than 100 normal human eyes. The wavefront refractions are obtained from an objective wavefront sensing and the manifest refractions are obtained from a subjective phoroptor.

FIG. 2 shows a conventional wavefront-guided vision correction with a mitigation of mismatches between the manifest refraction and the wavefront refraction. Wavefront-guided vision correction is allowed only if the difference of the spherical equivalent power between the manifest refraction and the wavefront refraction, |δSE|, is very small and within about 0.5 Dioptors. Wavefront-guided correction relies on the wavefront data from a wavefront sensor only.

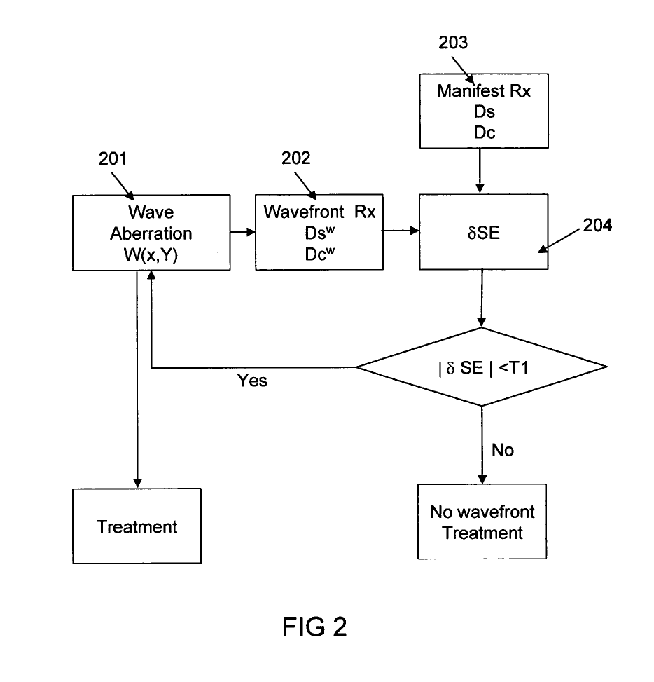

FIG. 3 shows a block diagram of an algorithm of wavefront fusion for determining a wave aberration of eye at its far accommodation point, based on a manifest refraction from a phoroptor and a wave aberration from a wavefront from wavefront sensing.

FIG. 4 shows a block diagram of a wavefront guided vision correction based on an Accommodation-Free Wavefront of eye. The accommodation-Free Wavefront is determined from the wavefront data from a wavefront sensor and a manifest refraction from the same eye.

FIG. 5 shows a block diagram of a True-Vision Wavefront of eye, based on an Accommodation-Free Wavefront and a refractive prescription if a conventional sphero-cylindrical correction is involved for vision correction.

### DETAILED DESCRIPTION OF THE INVENTION

Human eyes are dynamic optical systems with a variable focal length through accommodation. Refractive corrections are usually designed to achieve a best corrected image quality for an eye at the far accommodation point for the largest effective focus range possible.

Accommodation of an eye is well controlled to focus at the far accommodation point during a manifest refraction. It is ensured by setting an acuity chart at about 6 meters away from the tested eye and using an iterative approach to measure eye's visual acuity under different refractive corrections subjectively. Manifest refraction address the issue of accommodation and perceptional preference, but is limited for obtaining a sphero-cylindrical correction of focus error and cylindrical error only.

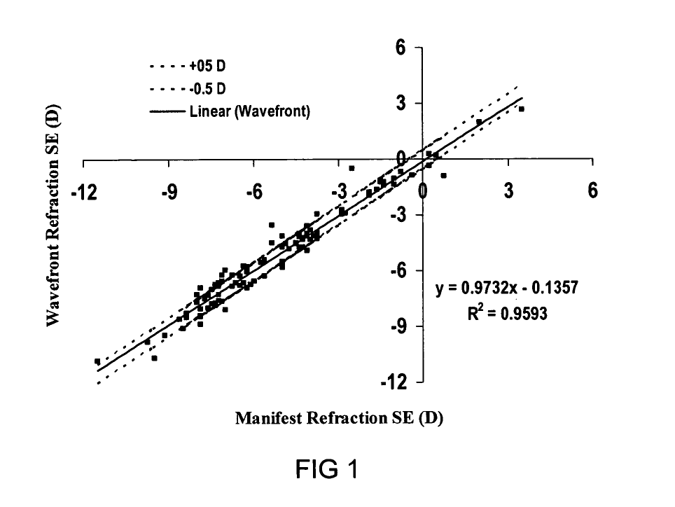

Accommodation of an eye is not fully controlled in conventional objective wavefront sensing. Wavefront aberrometers function just like an objective auto-refractor except that it has a capability for measuring high-order aberrations in the eye. The tested eye during a wavefront sensing can focus at a plan away from the far accommodation point, which is a main cause for the discrepancy between the manifest refraction and the wavefront refraction shown in FIG. 1. Wave aberration of an eye from wavefront sensing is thus not an Accommodation-Free Wavefront.

An algorithm of wavefront fusion is developed for determining an accommodation-free wavefront of an eye. The fusion algorithm provides an intelligent way to take advantages of the wavefront technology that measures all aberrations in the eye, and the manifest refraction that has been clinically effective for over a century for refractive vision correction.

Let us assume the wavefront error of an eye at the far accommodation point, or an Accommodation-Free Wavefront, is represented by WF(x,y). The Accommodation-Free Wavefront includes a conventional sphero-cylindrical error (focus and cylindrical errors) and a host of high-order aberrations.

A manifest refraction is known to provide a refractive prescription of an eye for a best sphero-cylindrical correction at the far accommodation point. Under a conventional refraction correction according to a manifest refraction, the uncorrected residual wave aberration in the eye at the far accommodation point (WFR) is
WFR=WF(x,y)−Ds(r)−Dc(x,y)−Ds^{b}(r), [1]
where Ds(r) and Dc(x,y) are the manifest spherical and cylindrical errors, respectively. x and y are Cartesian coordinates and r is the polar radius at the pupil of the eye. Ds^{b}(r) in Eq. 1 is a bias spherical power that represents a preference of individual opticians. Typically, the bias power Ds^{b} is small and can be ignored if the clinical preference is standardized.

With the removal of the physician bias power, the residual wavefront of an eye under a manifest correction takes the form of
WFR=WF(x,y)−Ds(r)−Dc(x,y). [2]
For an ideal eye without any high-order aberration, the residual wavefront (WFR) vanishes and the eye's wave aberration at the far accommodation point equals to the conventional sphero-cylindrical errors, i.e.,
WF(x,y)=Ds(r)+Dc(x,y). [3]
For normal human eyes having high-order aberrations, the accommodation-free wavefront (WF (x,y)) is
WF(x,y)=WFR+Ds(r)+Dc(x,y). [4]
Due to the inherent limitations, a manifest refraction does not provide neither the residual wavefront WFR nor the Accommodation-Free Wavefront WF(x,y).

Wavefront sensing measures all the aberrations in the eye objectively. From the measured wavefront W(x,y), we can find a best wavefront refraction that offers a best corrected image quality under a sphero-cylindrical correction. The residual wavefront of an eye with a wavefront sphero-cylindrical error removed takes the form of
WFR^{w}=W(x,y)−Ds^{w}(r)−Dc(x,y), [5]
where Ds^{w} and Dc^{w} are the wavefront refractions of the spherical and cylindrical errors, respectively.

The algorithm of wavefront fusion relies on the following four principles: 1) If focus error and astigmatism are the only aberrations corrected, the corrected eye under a manifest refraction has the best corrected optical quality at the far accommodation point. 2) Wave aberration from a wavefront sensing is a wavefront error at one accommodation state of the eye. 3) If focus error and astigmatism are the only aberrations corrected, the residual wave aberration under a wavefront sphero-cylindrical correction produces the best corrected optical quality for the eye at the accommodation state in the wavefront measurement. 4) The difference in the high-order aberrations of an eye at two different accommodation states is negligible if the change in the focus power of an eye between two accommodation states is small and within about 1.5 Dioptors.

From the fusion principle #1, we know that the uncorrected wave aberration WFR in Eq. 2 leads to the best corrected optical quality for an eye at its far accommodation point.

From the fusion principle #2 and #3, we know that the uncorrected wave aberration WFR^{w} in Eq. 5 leads to the best corrected optical quality for an eye at one accommodation state of eye during a wavefront measurement.

From the fusion principle #4, we know that the best corrected wavefront of an eye at the far accommodation point and the best corrected wavefront of the eye at the accommodation point of wavefront sensing is about the same if the accommodation offset between these two accommodation points is small and within an acceptable threshold.

If the accommodation point during a wavefront measurement is small within 1.5 Dioptors from the far accommodation point, we can reasonablely assume
WFR=WFR^{w}. [6]
Eq. 6 forms the bases for a wavefront fusion. From Eq. 2, Eq. 5 and Eq. 6, we obtain
W(x,y)−Ds^{w}(r)−Dc^{w}(x,y)=WF(x,y)−Ds(r)−Dc(x,y), [7]
and the wavefront of the eye at the far accommodation point WF(x,y) as
WF(x,y)=W(x,y)−Dc^{w}(x,y)−Ds^{w}(r)+Ds(r)+Dc(x,y). [8]
The Accommodation-Free Wavefront, WF(x,y), is a combination of a manifest refraction and a wave aberration from a wavefront sensing.

If we ignore the difference between the manifest and the wavefront cylindrical power, or Dc(x,y)=Dc^{w}(x,y), we obtain the accommodation-free wavefront of the eye as
WF(x,y)=W(x,y)+(Ds(r)−Ds^{w}(r)). [9]

Even though derived with the assumption that the cylindrical powers in the manifest and in the wavefront refractions are identical, the Accommodation-Free Wavefront in Eq. 9 is not constrained by this assumption of the cylindrical powers. Because the wavefront in Eq. 9 does not contain any cylindrical power, the Accommodation-Free Wavefront in Eq. 9 will neither be affected by the accuracy in the manifest cylindrical power nor by the calculation of the wavefront cylindrical power.

The Accommodation-Free Wavefront in Eq. 9 can be rewritten as the measured wave aberration of the eye W(x,y) plus an accommodation offset Φsⁱ(r), i.e.,
WF(x,y)=W(x,y)+Φsⁱ(r), [10]
where the accommodation offset Φsⁱ(r) equals to the difference between the manifest spherical power and the wavefront spherical power, i.e.,
Φsⁱ(r)=Ds(r)−Ds^{w}(r). [11]

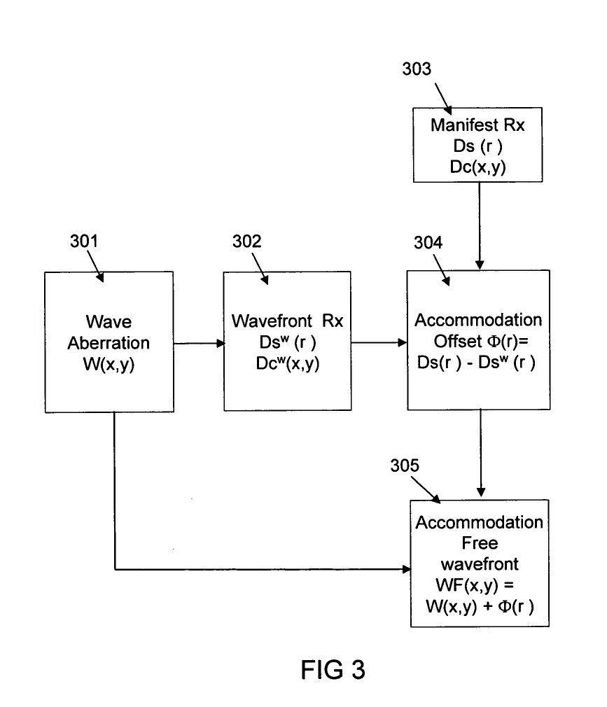

FIG. 3 shows a block diagram of the fusion algorithm for determining an accommodation-free wavefront of the eye. First, wave aberration of an eye 301 is measured with a wavefront sensor for the eye and wavefront refractions of sphero-cylindrical errors 302 are determined. The wavefront of the eye with a correction of wavefront sphero-cylindrical correction produces a best corrected optical quality for the eye. Second, a conventional manifest refraction of the same eye 303 at the far accommodation point is measured with a conventional phoroptor with a acuity chart about 6 meters away form the eye. Third, an accommodation offset of the eye 304 in a wavefront measurement is determined as the difference between the manifest spherical power and the wavefront spherical power. Fourth, the accommodation-free wavefront of the eye 305 is set as the wave aberration of the eye from a wavefront measurement plus an accommodation offset as the difference between the manifest spherical power and the wavefront spherical power.

Different from conventional treatments of wavefront data, we assume that wavefront sensors measure a wave aberration of an eye at one accommodation state of eye. If the accommodation offset of wavefront sensing, measured by (Ds(r)−Ds^{w}(r)), is small and less than about 1.5 Dioptors, we can obtain the accommodation-free wavefront of the eye from the measured wave aberration W(x,y) and the determined accommodation offset.

If the manifest spherical power equals to the wavefront spherical power of wavefront sensing, the wavefront sensor measures wave aberration of the eye at its far point, i.e.,
WF(x,y)=W(x,y). [12]

If an eye is emmetropic according to manifest refraction but is myopic of −1.0 Dioptor according to a wavefront measurement, the Accommodation-Free Wavefront is
WF(x,y)=W(x,y)+1.0 D. [13]
By adding the accommodation offset of 1.0 Dipotor to the wavefront from wavefront sensing W(x,y), we make the Accommodation-Free Wavefront WF(x,y) emmetropic.

We must emphasize two important properties in using the Accommodation-Free Wavefront in Eq. 10. First, the accommodation offsets Φsⁱ(r) should be within a small range so that the difference in the high-order aberrations at two different accommodation states is negligible. In extreme cases when the accommodation offset Φsⁱ(r) is large enough to cause a significant change in spherical aberration, an addition of spherical aberration to the accommodation-free wavefront in Eq. 10 may be necessary. The amount of added spherical aberration depends on the magnitude of the accommodation offset. Second, an additional −⅙ Dioptors can be added to the manifest refraction because vision charts are often set at 6 meter away instead of at infinity from the tested subjects.

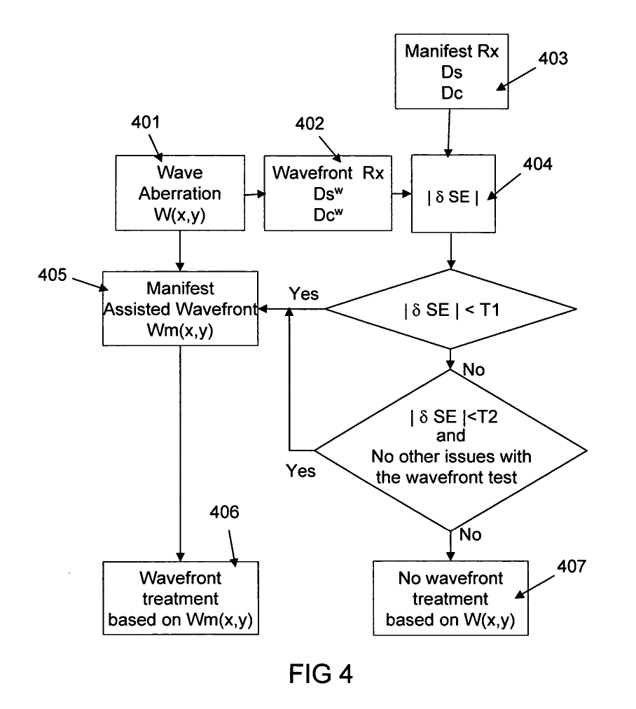

Determining an accommodation-free wavefront of an eye will enable improved wavefront-guided vision corrections as well as for reliable vision diagnosis because refractive corrections are usually designed to achieve a best corrected image quality for an eye at the far accommodation point. FIG. 4 shows a block diagram of an Accommodation-Free Wavefront-guide vision correction. A preferred embodiment is described below:

First, wave aberration of the eye (W(x,y)) 401 is measured by a wavefront sensor, and the wavefront refractions (Ds^{w} and Dc^{w}) 402 are determined using an algorithm that offers the best image quality for the eye when the wavefront refractions are removed.

Second, spherical equivalent power of the eye, SE, is determined from the manifest refraction 403 and from the wavefront refraction 402. The absolute difference between the spherical equivalent powers (|δSE|) is calculated 404.

Third, different actions are taken based on the absolute difference between the spherical equivalent powers (|δSE|). a) If |δSE| is less than a threshold value T1 (e.g., about 0.5D or one standard deviation for the difference between the manifest and the wavefront spherical equivalents in a normal population), a modified Wm(x,y) 405 is determined for the Accommodation-Free wavefront-guided vision correction. A wavefront-guided treatment 406 is performed based on the modified wavefront Wm(x,y). b) If |δSE| is beyond the threshold value T1 but less than the threshold T2 (e.g., 1.5D or 3 times the standard deviation for the difference between the manifest and the wavefront spherical equivalents in a population), the raw wavefront data should be reviewed. If there is no other known issues with the wavefront measurement (e.g., mis-identified raw data), a modified wavefront Wm(x,y) 405 can be determined and used for the Accommodation-Free wavefront-guided vision correction. A wavefront-guided treatment 406 is performed based on the modified wavefront Wm(x,y). c) No wavefront-guide vision correction 407 may be performed based on the wavefront data if |δSE| is larger than the threshold value T2 (about 1.5 Dioptors) or issues were found in reviewing the raw wavefront data (e.g., mis-identified raw data).

One preferred embodiment of an accommodation-free wavefront from the wavefront sensors is the wavefront Eq 10, i.e.,
Wm(x,y)=W(x,y)+(Ds(r)−Ds^{w}(r)). [14]
The accommodation offset can also takes a more general form with which the accommodation-free wavefront of the eye is
Wm(x,y)=W(x,y)+function(Ds, Ds^{w}), [15]
where the accommodation offset is a general function of the manifest spherical power (Ds) and the wavefront spherical power (Ds^{w}). In some embodiments, the accommodation offset can also be obtained from Eq. 8, depending not only on the spherical powers but also on the cylindrical powers. A general form of accommodation-free wavefront takes form of
Wm(x,y)=W(x,y)+function(Ds, D^{w}s, Dc, D^{w}c). [16]

The algorithm of wavefront fusion solves the problem of accommodation of eyes in wavefront sensing and makes it no longer necessary to match the wavefont refraction to the manifest refractions tightly. By relaxing the threshold for the difference in manifest and wavefront refraction, Accommodation-Free wavefront makes about 98% eyes wavefront treatable (within three times the standard deviation) instead of about 70% treatable (within one standard deviation) for the same wavefront technology. Accommodation-Free Wavefront can also reduce re-treatment by avoiding under-corrections or over-corrections caused by the accommodation offset in wavefront sensing when only wavefront data from wavefront sensors alone are used for vision corrections. Accommodation-Free Wavefront can be applied to all wavefront-guided vision corrections including wavefront-guided laser vision corrections, wavefront-guided contact lenses, wavefront-guided spectacles and wavefront-guided intro-ocular lenses.

As mentioned in the background, determining an Accommodation-Free Wavefront is also essential for reliable vision diagnosis. Two wavefront forms are commonly used in conventional vision evaluations: the original wavefront W(x,y) from wavefront sensing and the best-corrected wavefront under a best sphero-cylindrical correction WFR^{w} in Eq. 5.

Neither the original wavefront W(x,y) nor the best corrected wavefront WFR^{w} in Eq. 5 is suited for evaluating eyes reliably. First, the measured wavefront W(x,y) from a wavefront aberrometer is not the true wavefront of the eye at its far accommodation points because of a possible accommodation offset in a wavefront measurement. Second, the wavefront under a best wavefront sphero-cylindrical correction in Eq. 5
WFR^{w}=W(x,y)−Ds^{w}(r)−Dc^{w}(x,y),
is theoretical and rarely achieved in real life because Ds^{w}(r) and Dc^{w}(x,y) are theoretical corrections. True-vision wavefront can only be obtained with wavefront of an eye at the far accommodation point and with the true prescriptions of correction lenses used in vision corrections.

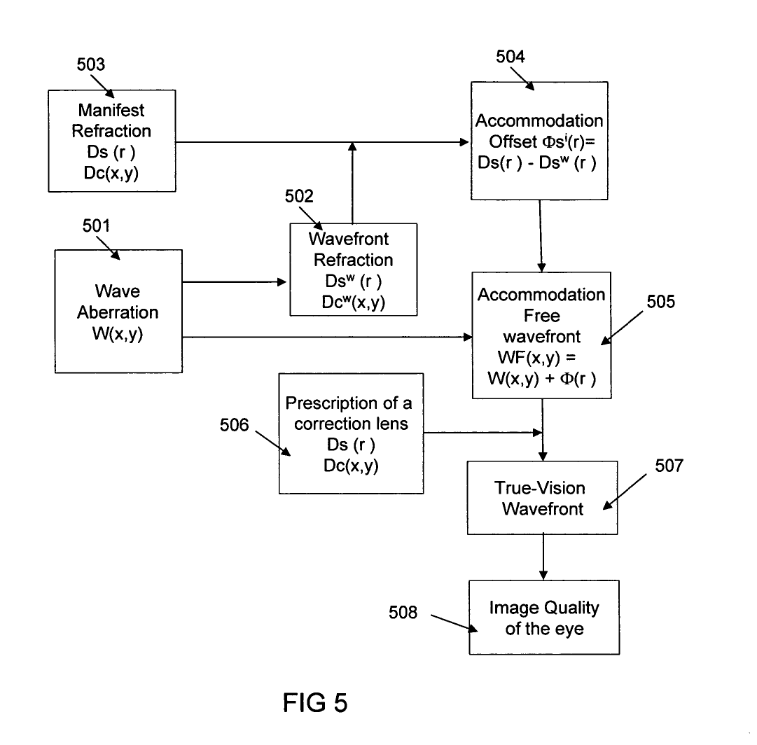

Based on the algorithm of wavefront fusion for the Accommodation-Free Wavefront, we propose a True-Vision Wavefront (TWF) of an eye for reliable vision diagnosis. FIG. 5 shows a block diagram of a True-Vision Wavefront of eye based on an Accommodation-Free Wavefront and a refractive prescription. First, wave aberration of an eye 501 is measured with a wavefront sensor for the eye and wavefront refractions of sphero-cylindrical errors 502 are calculated. The wavefront of the eye with a correction of wavefront sphero-cylindrical correction produces a best corrected optical quality for the eye. Second, conventional manifest refraction of the same eye 503 is measured with a conventional phoroptor with an acuity chart about 6 meters away form the eye. Third, an accommodation offset of the eye 504 during the wavefront measurement from the eye's far point is determine as the difference between the manifest spherical power and the wavefront spherical power. Fourth, the accommodation-free wavefront of the eye 505 is set as the wave aberration of the eye from a wavefront measurement plus an accommodation offset as the difference between the manifest spherical power and the wavefront spherical power. Fifth, true-vision wavefront of the eye 507 is determined. If no conventional sphero-cylindrical correction is used in real life, The True-Vision Wavefront of an eye is the accommodation-free wavefront 505. If a conventional sphero-cylindrical correction is used in real life, the true-vision wavefront of the eye 507 is obtained by combining the accommodation-free wavefront 505 and the prescription of the correction lens 506. Sixth, vision diagnosis of the eye can be obtained based on the true-vision wavefront 508.

The True-Vision Wavefront offers the most realistic wavefront for the evaluation of eye's image quality because it is based on the wave aberration of the eye at far accommodation point and the prescription of a true correction lens. Two categories of True-Vision Wavefronts are described. First, if no conventional sphero-cylindrical correction is used in real life, The True-Vision Wavefront of an eye is the accommodation-free wavefront
TWF=WF(x,y),
or
TWF=W(x,y)+(Ds(r)−Ds^{w}(r)), [17]
Second, if a conventional sphero-cylindrical correction is used in real life, the Corrected True-Vision Wavefront (CTWF) of a corrected eye is
CTWF(x,y)=TWF=(Ds(r)+Dc(x,y)), [18]
or
CTWF(x,y)=W(x,y)+(Ds(r)−Ds^{w}(r))−(Ds(r)+Dc(x,y))=W(x,y)−Ds^{w}(r)−Dc(x,y). [19]

For an emmetropic eye, or an eye having a visual acuity of 20/20 or better without any vision correction (Ds(r)=0), the True-Vision Wavefront is
TWF=W(x,y)−Ds^{w}(r). [20]
where W(x,y) is the wavefront from a wavefront sensing, Ds^{w}(r) being the spherical power in the wavefront refraction. TWF in Eq. 20 is a true-vision wavefront of an emmetropic eye because the cylindrical error in emmetropic eyes is not corrected in the real life while the spherical power is perfectly corrected through accommodation. If the cylindrical error in an emmetropic eye is larger enough, an addition of a balance spherical power can be introduced in the True-Vision Wavefront in Eq. 20.

Myopic eyes require a vision correction with negative lenses to achieve a visual acuity of 20/20 or better. A myopic eye without a vision correction has the True-Vision wavefront is the Accommodation-Free Wavefront, i.e.,
TWF=W(x,y)+(Ds(r)−Ds^{w}(r)). [21]
where W(x,y) is the wavefront obtained from a wavefront sensing, Ds^{w} (r) being the spherical power in wavefront refraction, and Ds(r) being the manifest spherical power of the eye.

For a myopic eye with a real sphero-cylindrical correction in life, the corrected True-Vision Wavefront is, according to Eq. 19,
WF(x,y)=W(x,y)−Ds^{w}(r)−Dc(x,y) [22]
where W(x,y) is the wavefront from a wavefront sensing, Ds^{w}(r) being the wavefront spherical power in the wavefront refraction, and Dc(x,y) being the prescribed cylindrical power of the correction lens.

Hyperopic eye requires a vision correction with positive lenses to achieve a visual acuity of 20/20 or better. For a low-hyperopic eye without a need of a refractive correction, the True-Vision Wavefront is the same as that of the emmetropic eye in Equation [20]. For a high-hyperopic eye with a real sphero-cylindrical correction, the True-Vision Wavefront is the same as that of the myopic eyes in Equation [22].

The True-Vision Wavefront in Eq. 17 through Eq. 22 provides more realistic wavefront than the original wavefront W(x,y) and the perfectly corrected wavefront in Eq. 5 for the evaluation of vision in real eyes.

Having obtained the True-Vision wavefront, we can derive vision performances of human eyes such as point-spread functions, modulation transfer functions, scores of night vision symptoms, and the optics-limited acuity for vision screening of naked eye, for comprehensive diagnosis of symptomatic eyes, and for performance evaluation and specification of various vision corrections.

A number of embodiments have been described. Nevertheless, it will be understood that various modifications may be made without departing from the spirit and scope of the invention. For example, advantageous results still could be achieved if steps of the disclosed techniques were performed in a different order and/or if components in the disclosed systems were combined in a different manner and/or replaced or supplemented by other components. Accordingly, other embodiments are within the scope of the following claims.

What is claimed is:

1. A method of wavefront fusion for determining an wavefront of an eye at its far accommodation point, the method comprising:
obtaining a wave aberration of an eye from a wavefront measurement;
obtaining a manifest refraction of an eye at the far accommodation state;
determining a wave aberration of the eye at its far accommodation point based on a combination of the manifest refraction and the measured wave aberration of the same eye.

2. The method of claim 1, wherein obtaining a wave aberration of an eye from a wavefront measurement comprises measuring at least one wave aberration of an eye with a wavefront aberrometer, including a Hartmann-Shack sensor based-aberrometer.

3. The method of claim 2, wherein the measured wave aberration of an eye comprises a wavefront sphero-cylindrical correction (a spherical power and a cylindrical power) and high-order aberrations in the eye.

4. The method of claim 1, wherein obtaining a manifest refraction of an eye at the far accommodation state comprises:
setting a resolution chart at a distance around 6 meters away from the tested eye;
changing refractive corrections of spherical power, cylindrical power and cylindrical axis for the tested eye;
determining a manifest refraction for the eye at the far point based on subjective feedbacks of the tested patient using a recursive process.

5. The method in claim 1, wherein the manifest refraction comprises at least a spherical power.

6. The method of claim 1, wherein the manifest refraction is measure at small pupil sizes around 3 mm for reduced focus depth.

7. The method of claim 1, wherein determining a wave aberration of the eye at its far accommodation point based on a combination of the manifest refraction and the measured wave aberration of the same eye comprises adding an accommodation offset, which equals to the difference between the manifest spherical power and the wavefront spherical power, to the obtained wave aberration of the eye.

8. The method of claim 7, wherein the wave aberration of the eye at the far accommodation point further includes an addition of Seidel spherical aberration with a magnitude depending on the difference between the manifest spherical power and the wavefront spherical power.

9. A method of accommodation-free wavefront-guided vision corrections comprises:
obtaining a wave aberration of an eye from a wavefront measurement;
obtaining a manifest refraction of an eye at the far accommodation state;
determining an accommodation-free wavefront of the eye based on a combination of the manifest refraction and the wave aberration of the eye;
correcting an optical error of an eye based on the accommodation-free wavefront.

10. The method of claim 9, wherein obtaining a wave aberration of an eye from a wavefront measurement comprises measuring at least one wave aberration of an eye with a wavefront aberrometer, including a Hartmann-Shack sensor based-aberrometer.

11. The method of claim 10, wherein the measured wave aberration of an eye comprises a wavefront sphero-cylindrical correction (a spherical power and a cylindrical power) and high-order aberrations in the eye.

12. The method of claim 11, wherein the wavefront sphero-cylindrical correction is determined to produce a best corrected optical quality for the eye under a conventional sphero-cylindrical correction.

13. The method of claim 9, wherein obtaining a manifest refraction of an eye at the far accommodation state comprises:
setting a resolution chart at a distance around 6 meters away from the tested eye;
changing refractive corrections of spherical power, cylindrical power and cylindrical axis for the tested eye;
determining a manifest refraction for the eye at a far point based on subjective feedbacks of the tested patient using a recursive process.

14. The method of claim 9, wherein determining an accommodation-free wavefront of the eye based on a combination of the manifest refraction and the wave aberration of the eye comprises adding an accommodation offset, which equals to the difference between the manifest spherical power and the wavefront spherical power, to the obtained wave aberration of the eye.

15. The method of claim 14, further comprises a condition that the accommodation offset, or the difference between the manifest spherical power and the wavefront spherical power, is within a pre-determined limit.

16. The method of claim 15, wherein the pre-determined limit is about 1.5 Dioptors.

17. The method of claim 14, wherein the wave aberration of the eye at the far accommodation point further includes an addition of Seidel spherical aberration with a magnitude depending on the difference between the manifest spherical power and the wavefront spherical power.

18. The method of claim 9, wherein an accommodation-free wavefront of the eye, or eye's wave aberration of the eye at its far accommodation point, is obtained from the measured wave aberration of the eye by replacing the wavefront sphero-cylindrical correction with the manifest sphero-cylindrical correction if the absolute difference between the manifest spherical power and wavefront spherical power is less than about 1.5 Diopters.

19. The method of claim 9, wherein correcting an optical error of an eye based on the accommodation-free wavefront comprises a procedure of wavefront-guided laser vision correction.

20. The method of claim 19, further comprising
a processor for generating an ablation pattern of laser energy for ablation of a corneal tissue of the eye so as to correct the measured optical error, the ablation pattern based at least in part on an accommodation-free wavefront; and
a laser system for directing laser energy onto the corneal tissue of the eye to achieve the generated ablation pattern.

21. The method of claim 9, wherein correcting an optical error of an eye based on the accommodation-free wavefront comprises a vision correction with a wavefront-guided spectacle.

22. The method of claim 21, further comprises
a system for producing a wavefront-guided spectacles so as to correct the measured an optical error in an eye, based at least in part on the accommodation-free wavefront.

23. The method of claim 9, wherein correcting an optical error of an eye based on the accommodation-free wavefront comprises a vision correction with a wavefront-guided contact lens or a wavefront-guided intro-ocular lens.

24. The method of claim 23, further comprises
a system for producing a wavefront-guided contact lenses or intro-ocular lenses so as to correct the measured an optical error in the eye, based at least in part on the accommodation-free wavefront.

25. a method of comprehensive vision diagnosis based on a true-vision wavefront, the method comprising:
obtaining a wave aberration of an eye from a wavefront measurement;
obtaining a manifest refractive correction at the far accommodation point if the eye is myopic or hyperopic;
obtaining a refractive prescription of a true correction lens if a conventional sphero-cylindrical correction is involved for a refractive correction;
calculating an true-vision wavefront based on the measured wave aberration, the manifest refraction, and the refractive prescription if a correction lens is involved;
calculating at least one vision performance parameter based on the true-vision wavefront of the eye for refractive vision diagnosis.

26. The method of claim 25, wherein obtaining a wave aberration of an eye from a wavefront measurement comprising measuring at least one wave aberration using a wavefront aberrometer, including a Hartmann-Shack sensor-based aberrometer.

27. The method of claim 25, wherein the measured wave aberration of an eye comprises a wavefront sphero-cylindrical correction and high-order aberrations in the eye.

28. The method of claim 27, wherein the wavefront sphero-cylindrical correction is determined to produce a best corrected optical quality for the eye when only defocus and astigmatism are corrected.

29. The method of claim 25, wherein the manifest refractive correction comprise zero refractive power for an emmetropic eye without vision correction.

30. The method of claim 29, wherein the true-vision wavefront is the measured wave aberration of the eye with removal of the wavefront spherical power.

31. The method of claim 25, wherein calculating an true-vision wavefront based on the measured wave aberration, the manifest refraction, and the refractive prescription if a correction lens is involved comprises:
calculating an accommodation free-wavefront as the measured wave aberration with addition of an accommodation offset that equals to the difference between the manifest spherical power and the wavefront spherical power;
calculating a true-vision wavefront from the accommodation-free wavefront with removal of a refractive prescription of a true correction lens if the lens is used for a sphero-cylindrical correction.

32. The method of claim 25, wherein calculating at least one vision performance parameter based on the true-vision wavefront of the eye comprises calculating at least one root-squares mean (RMS) wavefront error from the true-vision wavefront.

33. The method of claim 25, wherein calculating at least one vision performance parameter based on the true-vision wavefront of the eye comprises calculating at least one point-spread function of the eye from the true-vision wavefront.

34. The method of claim 25, wherein calculating at least one vision performance parameter based on the true-vision wavefront of the eye comprise:
calculating at least one point-spread function of the eye from the true-vision wavefront;
calculating at least one retinal image in the eye by convolving a point-spread function with a vision chart for vision diagnosis of visual acuity and aberration-induced vision symptoms such as glare, halo, ghost image, and starburst.

35. The method of claim 25, wherein calculating at least one vision performance parameter based on the true-vision wavefront of the eye comprises calculating at least one modulation-transfer function of the eye from the true-vision wavefront.

36. The method of claim 35, wherein the modulation transfer function is represented by a relative MTF score for vision clarity, determined from modulation transfer function of the tested eye and modulation-transfer functions of a cohort of eyes with normal visual acuity.
